(19) United States
(12) Patent Application Publication
(10) Pub. No.: US 2021/0299413 A1
(43) Pub. Date: Sep. 30, 2021

(54) Torque device

(71) Applicant: TERUMO KABUSHIKI KAISHA, Tokyo (JP)

(72) Inventors: Daisuke SHIMADA, Fujinomiya-shi (JP); Takashi ITO, Fujinomiya-shi (JP); Shouta ARAHIRA, Fujinomiya-shi (JP)

(73) Assignee: TERUMO KABUSHIKI KAISHA, Tokyo (JP)

(21) Appl. No.: 17/346,720

(22) Filed: Jun. 14, 2021

Related U.S. Application Data
(63) Continuation of application No. PCT/JP2020/009969, filed on Mar. 9, 2020.

(30) Foreign Application Priority Data
Mar. 13, 2019 (JP) 2019-045466

(51) Int. Cl. A61M 25/09

(52) U.S. Cl. CPC A61M 25/09

(57) ABSTRACT

A torque device that has good manual operability and rather easily attaches to an elongated body such as a guide wire. A torque device for holding an elongated body comprises a first member and a second member in which is formed a connected space through which the elongated body can be inserted in the length direction. The first member and the second member are able to rotate with each other in a state in which the elongated body is being held, and have an opening that allows the space to communicate with the outside in the radial direction. At least one of the first member and the second member has a diameter decreasing portion that reduces the diameter of the space as a result of the first member and the second member rotating with each another.

### CROSS-REFERENCES TO RELATED APPLICATIONS

This application is a continuation of International Application No. PCT/JP2020/009969 filed on Mar. 9, 2020, which claims priority to Japanese Patent Application No. 2019-045466 filed on Mar. 13, 2019, the entire content both of which is incorporated herein by reference.

### FIELD OF THE DISCLOSURE

The present disclosure relates to a torque device that helps improve the operability of an elongated body such as a guide wire inserted into a living body.

### BACKGROUND DISCUSSION

When a catheter is inserted into a biological lumen, a guide wire is used as an elongated body that guides the catheter to a target site in the biological lumen. The guide wire is inserted along an axial direction of the catheter, and is inserted into the biological lumen prior to the catheter to guide the insertion of the catheter. The guide wire is also used to guide an endoscope. Since the biological lumen is bent and branched, it is necessary to move the guide wire forward and backward and rotate the guide wire when inserting the guide wire into the biological lumen.

Since the guide wire has a relatively small outer diameter, the guide wire can be difficult to hold and operate. Therefore, there is known a torque device attached to a hand portion of the guide wire to facilitate operation. Examples of the torque device include those described in Japanese Patent Application Publication No. 2011-62522 A.

### SUMMARY

A torque device is disclosed that can be rather easily attached to an elongated body such as a guide wire and has good manual operability.

A torque device that holds an elongated body according to the disclosure includes: a first member and a second member that define a continuous space through which the elongated body is inserted along a longitudinal direction, in which the first member and the second member are rotatable relative to each other while holding the elongated body, and have an opening that connects the space and outside in a radial direction, the opening is continuous in a length direction of the first member and the second member, and has a width through which the elongated body is insertable, and at least one of the first member and the second member includes a diameter decreasing portion that decreases a diameter of the space by the first member and the second member rotating relative to each other.

The torque device as disclosed can rather easily guide the elongated body from the opening to the space, and is configured to hold the elongated body disposed in the space by the first member and the second member rotating relative to each other, and therefore, the elongated body can be rather easily attached and held. Further, the elongated body attached and held by the torque device can be moved forward and backward and rotated, and the operability can be improved.

The first member and the second member may move along the length direction of the elongated body by rotating relative to each other, and the diameter decreasing portion of one of the first member and the second member may be decreased in diameter by the other one of the first member and the second member moving along the length direction of the elongated body. Accordingly, it is possible to form a structure in which the diameter decreasing portion is decreased with a simple structure of forming a screw surface on the first member and the second member.

The diameter decreasing portion may be elastically deformable in the radial direction, and may have a pressing surface that is pressed in the radial direction by the other one of the first member and the second member when the first member and the second member move along the longitudinal direction of the elongated body by rotating relative to each other. Accordingly, the elongated body can be elastically held by the diameter decreasing portion, and the operability of the held elongated body can be further improved.

At least one of the first member and the second member may have an elastically deformable inner peripheral member that is in pressure contact with or joined to the other one of the first member and the second member, and the inner peripheral member may have an inner peripheral surface that constitutes at least a part of a peripheral surface of the space to form the diameter decreasing portion. Accordingly, by the first member and the second member rotating relative to each other, it is possible to form a structure in which the inner peripheral member is twisted to decrease the diameter.

The inner peripheral member provided on one of the first member and the second member may include a protruding portion that enters the other one of the first member and the second member. Accordingly, the inner peripheral surface of the inner peripheral member can be enlarged, and the elongated body can be held rather firmly.

The opening may be continuous in the longitudinal direction of the elongated body over the first member and the second member in a state where the diameter decreasing portion is enlarged. Accordingly, the elongated body can be rather easily inserted into the space through the opening.

In accordance with an aspect, a torque device is disclosed comprising: a first member and a second member having a continuous space configured to receive a guide wire along a longitudinal direction of the first member and the second member; the first member and the second member configured to be rotatable relative to each other while holding the guide wire and movable along a longitudinal direction of the guide wire by the rotation relative to each other; and at least one of the first member and the second member includes a diameter decreasing portion that decreases a diameter of the space by the first member and the second member rotating relative to each other, and the diameter decreasing portion of one of the first member and the second member is configured to decrease in diameter by the other one of the first member and the second member moving along the longitudinal direction of the guide wire.

In accordance with another aspect, a method is disclosed of holding an elongated body, the method comprising: inserting an elongated body through an opening into a continuous space of a torque device, the torque device including a first member and a second member that define the continuous space along a longitudinal direction of the first member and the second member, the first member and the second member includes the opening that connects the space and outside in a radial direction, the opening being continuous in the longitudinal direction of the first member and the second member, and at least one of the first member and the second member includes a diameter decreasing portion; and holding the elongated body in the torque device by rotating the first member and the second member relative to each other in a first direction to decrease a diameter of the space with the diameter decreasing portion of the at least one of the first member and the second member.

### BRIEF DESCRIPTION OF THE DRAWINGS

FIG. 1 is a perspective view of a torque device according to a first exemplary embodiment.

FIG. 2 is a cross-sectional view of the torque device of FIG. 1.

FIG. 3 is a cross-sectional view of the torque device in a state where a diameter of a diameter decreasing portion is decreased.

FIG. 4 is a cross-sectional view of the torque device in a state where a guide wire is held by the diameter decreasing portion.

FIG. 5 is a cross-sectional view of a torque device according to a second exemplary embodiment.

FIG. 6 is a cross-sectional view of a torque device in a state where a diameter of a diameter decreasing portion of FIG. 5 is decreased.

FIG. 7 is an enlarged cross-sectional view of a part of a rotation latch mechanism.

FIG. 8 is a cross-sectional view of a torque device according to a third exemplary embodiment.

### DETAILED DESCRIPTION

Set forth below with reference to the accompanying drawings is a detailed description of embodiments of a torque device that helps improve the operability of an elongated body such as a guide wire inserted into a living body. Note that since embodiments described below are preferred specific examples of the present disclosure, although various technically preferable limitations are given, the scope of the present disclosure is not limited to the embodiments unless otherwise specified in the following descriptions. Note that dimensional ratios in the drawings may be exaggerated for convenience of description and may differ from the actual ratios.

A torque device 20 according to an embodiment of the disclosure is attached to a hand portion of a guide wire 10 to be inserted into a biological lumen to help improve the operability of the guide wire 10 by an operator.

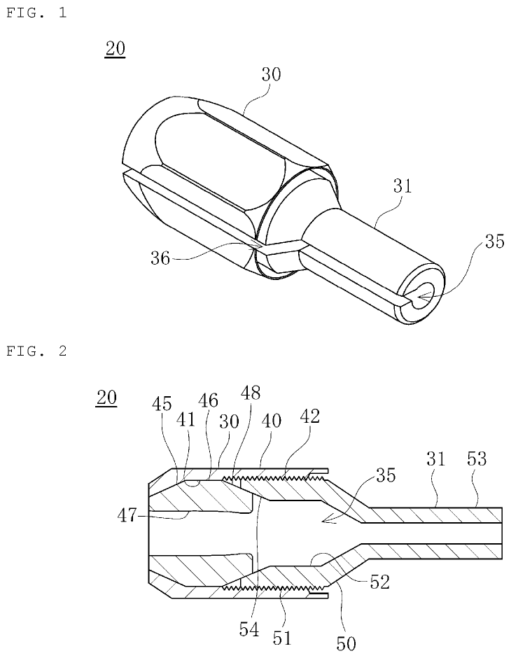

As shown in FIG. 1, the torque device 20 of the present embodiment includes a first member 30 and a second member 31 assembled with each other. That is, the first member 30 and the second member 31 may be connected to one another. Further, the torque device 20 may be formed with a continuous space 35 through the entire length of the torque device 20. The guide wire 10 can be inserted through the space 35 in a state where the first member 39 and the second member 31 are integrated. In addition, the torque device 20 has an opening 36 through the entire length of the torque device 20. The opening 36 connects the space 35 and the outside in a radial direction. The opening 36 has a width for the guide wire 10 to be inserted into the space 35 from the outside.

The first member 30 and the second member 31 are rotatable relative to each other and move relative to each other along a longitudinal direction (i.e., axial direction) with the rotation. By moving the first member 30 and the second member 31 relative to each other in the length direction, it is possible to hold the guide wire 10 inserted through the space 35 to be described later. The guide wire 10 can be introduced from the opening 36 into the space 35 in a state where the first member 30 and the second member 31 are disposed such that the opening 36 is continuous through the entire length of the torque device 20 as shown in FIG. 1.

As shown in FIG. 2, the first member 30 has an outer peripheral surface 40 facing the outside and an inner peripheral surface 41 facing the space 35. The inner peripheral surface 41 is formed with an inner peripheral screw surface 42 that is a screw thread for engaging an outer peripheral screw surface 51 of the second member 31.

The first member 30 includes a tubular diameter decreasing portion 45 on an end portion in the space 35. The diameter decreasing portion 45 can be made, for example, of an elastic material such as rubber. An outer peripheral surface 46 of the diameter decreasing portion 45 is abutted against the inner peripheral surface 41 of the first member 30. Further, an inner peripheral surface 47 of the diameter decreasing portion 45 has a diameter larger than that of the guide wire 10, and the guide wire 10 can be inserted in a state shown in FIGS. 1 and 2. Similar to the first member 30 and the second member 31, the diameter decreasing portion 45 has an opening (i.e., side opening) through the entire length of the diameter decreasing portion 45 (i.e., distal end to a proximal end of the diameter decreasing portion 45) in a circumferential direction, and the guide wire 10 can be inserted in the radial direction.

The second member 31 has an outer peripheral surface 50 having a diameter that substantially matches (i.e., approximately equal to) an inner diameter of the first member 30. An outer diameter of the second member 31 on an end side exposed from the first member 30 is smaller than that of the outer peripheral surface 50 in the first member 30. The outer peripheral surface 50 of the second member 31 has an outer peripheral screw surface 51 that screws into the inner peripheral screw surface 42 of the first member 30.

The second member 31 is formed with a tapered surface 54 at an end portion in the first member 30. The tapered surface 54 has an inner diameter increasing toward an end surface side. The tapered surface 54 is abutted against a pressing surface 48 that is an end portion outer peripheral surface of the diameter decreasing portion 45. The pressing surface 48 of the diameter decreasing portion 45 is inclined in the same direction as the tapered surface 54, and is pressed (i.e., moved) toward an inner peripheral side by the tapered surface 54 when the pressing surface 48 moves in the longitudinal direction of the guide wire 10.

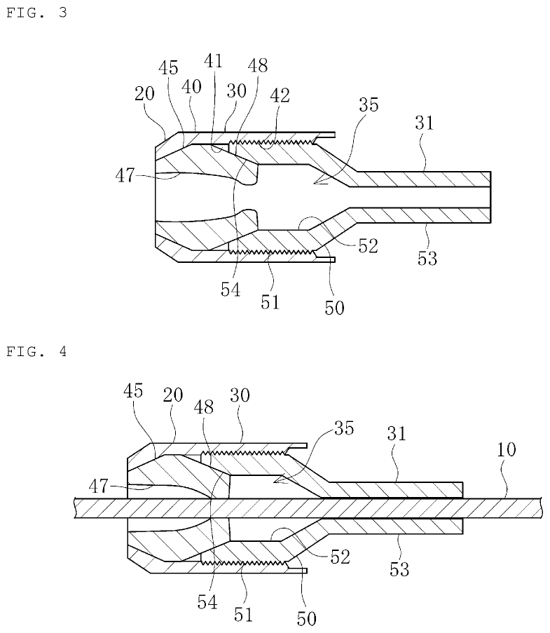

As shown in FIG. 3, when the first member 30 and the second member 31 are rotated relative to each other and the second member 31 is moved in the longitudinal direction to enter the inside of the first member 30, the pressing surface 48 of the diameter decreasing portion 45 is pressed toward the inner peripheral side by the tapered surface 54 of the second member 31. Accordingly, the diameter decreasing portion 45 is deformed toward the inner peripheral side, and the inner diameter becomes smaller.

As shown in FIG. 4, when the first member 30 and the second member 31 are rotated relative to each other in a state where the guide wire 10 is inserted, the guide wire 10 is held by the diameter decreasing portion 45. Accordingly, the guide wire 10 can be fixed in the longitudinal direction and the radial direction with respect to the torque device 20, and the operator can move the guide wire 10 forward and backward and can rotate the guide wire 10 via the torque device 20.

When the torque device 20 is used, a positional relationship between the first member 30 and the second member 31 is set to the state shown in FIG. 2 in advance. In this state, the opening 36 is continuous over the first member 30 and the second member 31 as shown in FIG. 1, and the guide wire 10 can be inserted into the space 35 through the opening 36. After the guide wire 10 is inserted into the space 35, a position of the torque device 20 in the longitudinal direction of the guide wire 10 can be adjusted. After the position of the torque device 20 is determined, the first member 30 and the second member 31 are rotated relative to each other into a state shown in FIG. 4. Accordingly, the guide wire 10 of the space 35 is held by the diameter decreasing portion 45. When the guide wire 10 is held by the diameter decreasing portion 45, the guide wire 10 can be moved forward and backward and rotated via the torque device 20.

When the torque device 20 is removed from the guide wire 10, the first member 30 and the second member 31 are rotated relative to each other to change from the state of FIG. 4 to the state of FIG. 2. Accordingly, a state of the diameter decreasing portion 45 of holding the guide wire 10 is released, and the guide wire 10 can be removed from the space 35 to the outside through the opening 36.

As described above, in the torque device 20 of the present embodiment, the guide wire 10 can be inserted in the radial direction from the opening 36 which is continuous over the first member 30 and the second member 31, and the inserted guide wire 10 can be held by the diameter decreasing portion 45 by rotating the first member 30 and the second member 31 relative to each other. Therefore, the guide wire 10 can be rather easily attached. Since the first member 30 and the second member 31 constituting the torque device 20 are rotary bodies, the torque device 20 holding the guide wire 10 can be rather easily rotated.

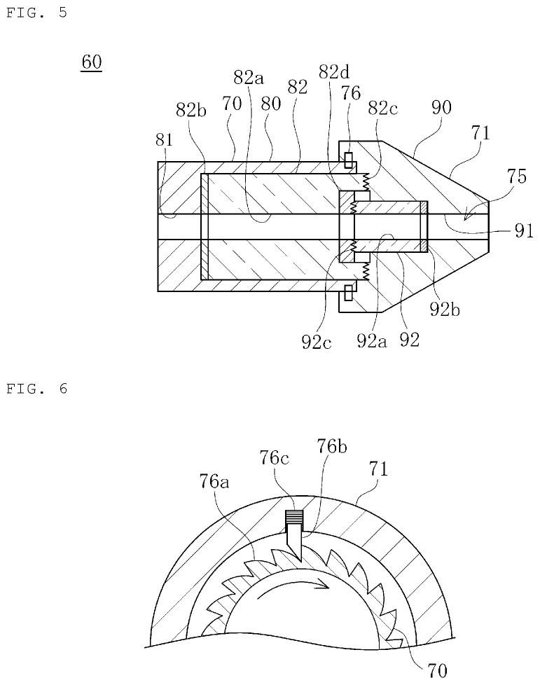

Next, a second exemplary embodiment of the disclosure will be described. As shown in FIG. 5, a torque device 60 of the second exemplary embodiment includes a first member 70 and a second member 71 that are rotatable relative to each other and a space 75 inside the torque device 60. The space 75 is continuous through the first member 70 and the second member 71. The guide wire 10 can be inserted through the space 75.

The torque device 60 is formed with an opening continuous over the first member 70 and the second member 71. The opening connects the space 75 and the outside in the radial direction as in the first exemplary embodiment.

The cylindrical first member 70 has an outer peripheral surface 80 and an inner peripheral surface 81, and a part of the inner peripheral surface 81 is formed by an inner peripheral member 82. The inner peripheral member 82 can be formed, for example, of an elastic material such as rubber, and an inner peripheral surface 82a constitutes a part of a peripheral surface of the space 75. One end of the inner peripheral member 82 in the length direction is a fixed portion 82b fixed to the first member 70, and the other end of the inner peripheral member 82 in the longitudinal direction is a pressure contact portion 82c that is in pressure contact with the second member 71. Further, on an inner peripheral side relative to the pressure contact portion 82c, the inner peripheral member 82 is provided with a hard portion 82d in pressure contact with an inner peripheral member 92 provided on the second member 71. The inner peripheral member 82 can be made, for example, of a relatively elastic and soft material, and the hard portion 82d is made of a relatively hard material.

The second member 71 has an outer peripheral surface 90 and an inner peripheral surface 91, and a part of the inner peripheral surface 91 is formed by the inner peripheral member 92. The inner peripheral member 92 can be formed of an elastic material, for example, such as rubber like the inner peripheral member 82 of the first member 70, and an inner peripheral surface 92a constitutes a part of the peripheral surface of the space 75. One end of the inner peripheral member 92 in the longitudinal direction can be a fixed portion 92b fixed to the second member 71, and the other end of the inner peripheral member 92 in the longitudinal direction (i.e., axial direction) is a pressure contact portion 92c that is in pressure contact with the hard portion 82c provided on the inner peripheral member 82 of the first member 70.

The first member 70 and the second member 71 can rotate relative to each other via a rotation latch 76. As shown in FIG. 6, the rotation latch 76 includes a gear 76a formed on the first member 70 and a claw 76b provided on the second member 71. The claw 76b is attached to the second member 71 via a biasing portion 76c, for example, such as a spring, and the claw 76b is biased in a direction of being pressed against the gear 76a. The gear 76a can include saw blade-shaped uneven portions along a circumferential direction, and each uneven portion can have a surface inclined along the circumferential direction and a surface substantially along the radial direction. The claw 76b can have an acute-angled tip, and one side of the claw 76b is substantially along the radial direction. Therefore, when the first member 70 rotates with respect to the second member 71 in a direction of an arrow in FIG. 6, the claw 76b can ride on the inclined surface of the gear 76a. When the first member 70 tries to rotate with respect to the second member 71 in a direction opposite to the arrow in FIG. 6, the side of the claw 76b that is substantially along the radial direction is abutted against a surface substantially along the radial direction of the gear 76a, and therefore, the first member 70 cannot rotate. Accordingly, the first member 70 can rotate only in one direction with respect to the second member 71 and the rotation state can be maintained.

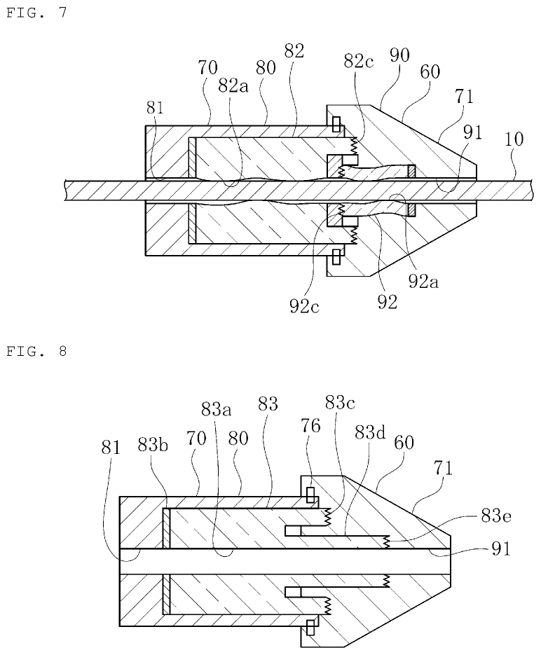

When the first member 70 and the second member 71 are rotated relative to each other, since both end of the inner peripheral member 82 of the first member 70 in the longitudinal direction are restrained correspondingly by the fixed portion 82b and the pressure contact portion 82c, twisting occurs along the length direction. Along with this, uneven portions are generated on the inner peripheral surface 82a of the inner peripheral member 82. Similarly, the inner peripheral member 92 of the second member 71 is twisted along the length direction, and uneven portions are generated on the inner peripheral surface 92a. Accordingly, as shown in FIG. 7, an inner diameter of the space 75 is decreased, and the guide wire 10 inserted into the space 75 can be held. That is, the inner peripheral members 82, 92 can function as diameter decreasing portions. Accordingly, the guide wire 10 is fixed in the longitudinal direction and the radial direction with respect to the torque device 60, and the operator can move the guide wire 10 forward and backward, and can rotate the guide wire 10 via the torque device 60.

Accordingly, the first member 70 and the second member 71 are provided with the inner peripheral members 82, 92 whose both end portions in the longitudinal direction are restrained, respectively, and the first member 70 and the second member 71 are rotated relative to each other. Therefore, the guide wire 10 can be rather easily held by the rotary operation by twisting the inner peripheral members 82, 92 to decrease the diameter of the inner peripheral surfaces 82a, 92a.

Further, a configuration of the inner peripheral member 82, 92 is not limited to the embodiments disclosed above. For example, as shown in FIG. 8, an inner peripheral member 83 of the first member 70 has the pressure contact portion 82c that is in pressure contact with the second member 71 at an outer peripheral portion. Further, an inner peripheral surface 83a of the inner peripheral member 83 includes a protruding portion 83d that enters toward the second member 71. The protruding portion 83d enlarges the inner peripheral surface 83a of the inner peripheral member 83 to the inner peripheral surface 91 of the second member 71. A tip of the protruding portion 83d is a second pressure contact portion 83e that is in pressure contact with the second member 71. Accordingly, the inner peripheral member 83 provided on the first member 70 may have the inner peripheral surface 83a extending over the first member 70 and the second member 71. In FIG. 8, the inner peripheral member 83 is provided on the first member 70, and an inner peripheral member may be provided on the second member 71 such that an inner peripheral surface of the second member 71 enters the first member 70.

As described above, the torque device 20 according to the present embodiment that holds the elongated body 10 includes: the first member 30 and the second member 31 that define the continuous space 35 through which the elongated body 10 is inserted along the longitudinal direction. The first member 30 and the second member 31 are rotatable relative to each other while holding the elongated body 10, and have the opening 36 that connects the space 35 and the outside in the radial direction. The opening 36 is continuous in the longitudinal direction of the first member 30 and the second member 31, and has the width through which the elongated body 10 is insertable. At least one of the first member 30 and the second member 31 includes the diameter decreasing portion 45 that decreases the diameter of the space 35 by the first member 30 and the second member 31 rotating relative to each other. Accordingly, the torque device 20 according to the present embodiment can rather easily guide the elongated body 10 from the opening 36 to the space 35, and can hold the elongated body 10 disposed in the space 35 by the first member 30 and the second member 31 rotating relative to each other, and therefore, the elongated body 10 can be rather easily attached and held. Further, the elongated body 10 attached and held by the torque device 20 can be moved forward and backward and rotated, and the operability can be improved.

Further, the first member 30 and the second member 31 may move along the length direction of the elongated body 10 by rotating relative to each other, and the diameter decreasing portion 45 of one of the first member 30 and the second member 31 may be decreased in diameter by the other one of the first member 30 and the second member 31 moving along the length direction of the elongated body 10. Accordingly, it is possible to form a structure in which the diameter decreasing portion 45 is decreased with a simple structure of forming a screw surface on the first member 30 and the second member 31.

Further, the diameter decreasing portion 45 may be elastically deformable in the radial direction, and may have the pressing surface 48 that is pressed in the radial direction by the other one of the first member 30 and the second member 31 when the first member 30 and the second member 31 move along the longitudinal direction of the elongated body 10 by rotating relative to each other. Accordingly, the elongated body 10 can be elastically held by the diameter decreasing portion 45, and the operability of the held elongated body 10 can be further improved.

Further, at least one of the first member 70 and the second member 71 may have the elastically deformable inner peripheral member 82 that is in pressure contact with or joined to the other one of the first member 70 and the second member 71, and the inner peripheral member 82 may have the inner peripheral surface 82a that constitutes at least a part of the peripheral surface of the space 75 to form the diameter decreasing portion. Accordingly, by the first member 70 and the second member 71 rotating relative to each other, it is possible to form a structure in which the inner peripheral member 82 is twisted to decrease the diameter.

Further, the inner peripheral member 83 provided on one of the first member 70 and the second member 71 may have the protruding portion 83d that enters the other one of the first member 70 and the second member 71. Accordingly, the inner peripheral surface 83a of the inner peripheral member 83 can be enlarged and the elongated body 10 can be held relatively firmly.

Further, the opening 36 may be continuous in the length direction of the elongated body 10 over the first member 30 and the second member 31 in a state where the diameter decreasing portion 45 is enlarged. Accordingly, the elongated body 10 can be rather easily inserted into the space 35 through the opening 36.

Note that the disclosure is not limited to the embodiments described above and various modifications can be made by those skilled in the art within a scope of the technical idea of the disclosure. For example, in the above-described embodiment, the elongated body is the guide wire 10. Alternatively, the elongated body is not limited to the guide wire 10 as long as the elongated body is a member that is long in the longitudinal direction and is operated to move forward and backward and rotate and can withstand a pressure of the diameter decreasing portion 45.

Further, the first member 30 of the torque device 20 shown in FIG. 1 has a nut-shaped outer peripheral surface, and may have an outer peripheral surface having a shape other than the above shape as long as the first member 30 has a shape that is rather easy to operate.

Further, in the embodiments described above, the guide wire 10 is attached to the torque device 20 via the opening 36, and the guide wire 10 may be attached to the torque device 20 in advance. In this case, after moving the torque device 20 to any position in the length direction of the guide wire 10, the first member 30 and the second member 31 are rotated to hold the guide wire 10, and the guide wire 10 is operated via the torque device 20.

Further, in the torque device 60 in the second exemplary embodiment, the inner peripheral surfaces 82a, 92a of the inner peripheral members 82, 92 are relatively smooth surfaces, and protruding portions and uneven portions may be provided to make it easier to hold the guide wire 10.

The detailed description above describes embodiments of a torque device that helps improve the operability of an elongated body such as a guide wire inserted into a living body. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A torque device configured to hold an elongated body, the torque device comprising:
a first member and a second member that define a continuous space through which the elongated body is inserted along a longitudinal direction of the first member and the second member;
the first member and the second member configured to be rotatable relative to each other while holding the elongated body, and have an opening that connects the space and outside in a radial direction;
the opening is continuous in the longitudinal direction of the first member and the second member, and has a width through which the elongated body is insertable; and
at least one of the first member and the second member includes a diameter decreasing portion configured to decrease a diameter of the space by the first member and the second member rotating relative to each other.

2. The torque device according to claim 1, wherein
the first member and the second member are configured to move along a longitudinal direction of the elongated body by the rotation relative to each other; and
the diameter decreasing portion of one of the first member and the second member is decreased in diameter by the other one of the first member and the second member moving along the longitudinal direction of the elongated body.

3. The torque device according to claim 2, wherein the diameter decreasing portion is elastically deformable in the radial direction, and the diameter decreasing portion includes a pressing surface configured to be pressed in the radial direction by the other one of the first member and the second member when the first member and the second member move along the longitudinal direction of the elongated body by the rotation relative to each other.

4. The torque device according to claim 1, wherein at least one of the first member and the second member has an elastically deformable inner peripheral member configured to be in pressure contact with or joined to the other one of the first member and the second member, and the inner peripheral member has an inner peripheral surface that constitutes at least a part of a peripheral surface of the space to form the diameter decreasing portion.

5. The torque device according to claim 4, wherein the inner peripheral member provided on one of the first member and the second member includes a protruding portion configured to enter the other one of the first member and the second member.

6. The torque device according to claim 1, wherein the opening is continuous in the length direction of the elongated body over the first member and the second member in a state where the diameter decreasing portion is enlarged.

7. The torque device according to claim 1, wherein the first member has an outer peripheral surface facing the outside and an inner peripheral surface facing the space, and the inner peripheral surface has an inner peripheral screw surface configured to engage an outer peripheral screw surface of the second member.

8. The torque device according to claim 1, wherein an inner peripheral surface of the diameter decreasing portion has a diameter greater than a diameter of the elongated body.

9. The torque device according to claim 1, wherein the second member has an outer peripheral surface having an outer diameter that is approximately equal to an inner diameter of the first member, and an outer diameter of the second member on an end side exposed from the first member is smaller than the outer diameter of the outer peripheral surface of the first member, and the second member has a tapered surface having an inner diameter increasing toward an end surface at an end, and wherein the tapered surface is configured to abut against a pressing surface, the pressing surface being an end outer peripheral surface of the diameter decreasing portion.

10. The torque device according to claim 9, wherein the pressing surface of the diameter decreasing portion is inclined in a same direction as the tapered surface.

11. The torque device according to claim 1, wherein the elongated body is a guide wire.

12. The torque device according to claim 1, wherein the first member and the second member rotate relative to each other via a rotation latch, the rotation latch including a gear on the first member and a claw attached to the second member via a biasing portion, and the claw is biased in a direction of being pressed against the gear.

13. The torque device according to claim 12, wherein the gear includes saw blade-shaped uneven portions along a circumferential direction, and each uneven portion having a surface inclined along the circumferential direction and a surface substantially along the radial direction, and the claw has an acute-angled tip, and one side of the claw being substantially along the radial direction.

14. A torque device comprising:
a first member and a second member having a continuous space configured to receive a guide wire along a longitudinal direction of the first member and the second member;
the first member and the second member configured to be rotatable relative to each other while holding the guide wire and movable along a longitudinal direction of the guide wire by the rotation relative to each other; and
at least one of the first member and the second member includes a diameter decreasing portion that decreases a diameter of the space by the first member and the second member rotating relative to each other, and the diameter decreasing portion of one of the first member and the second member is configured to decrease in diameter by the other one of the first member and the second member moving along the longitudinal direction of the guide wire.

15. The torque device according to claim 14, wherein the diameter decreasing portion is elastically deformable in the radial direction, and the diameter decreasing portion includes a pressing surface configured to press in the radial direction by the other one of the first member and the second member when the first member and the second member move along the longitudinal direction of the guide wire by the rotation relative to each other.

16. The torque device according to claim 14, wherein at least one of the first member and the second member has an elastically deformable inner peripheral member configured to be in pressure contact with or joined to the other one of the first member and the second member, and the inner peripheral member has an inner peripheral surface that constitutes at least a part of a peripheral surface of the space to form the diameter decreasing portion, and the inner peripheral member is provided on one of the first member and the second member includes a protruding portion that enters the other one of the first member and the second member.

17. A method of holding an elongated body, the method comprising:
inserting an elongated body through an opening into a continuous space of a torque device, the torque device including a first member and a second member that define the continuous space along a longitudinal direction of the first member and the second member, the first member and the second member includes the opening that connects the space and outside in a radial direction, the opening being continuous in the longitudinal direction of the first member and the second member, and at least one of the first member and the second member includes a diameter decreasing portion; and
holding the elongated body in the torque device by rotating the first member and the second member relative to each other in a first direction to decrease a diameter of the space with the diameter decreasing portion of the at least one of the first member and the second member.

18. The method according to claim 17, further comprising:
moving the first member and the second member along a longitudinal direction of the elongated body by the rotation of the first member and the second member relative to each other; and
decreasing the diameter decreasing portion of one of the first member and the second member in diameter by the other one of the first member and the second member moving along the longitudinal direction of the elongated body.

19. The method according to claim 18, further comprising:
pressing a pressing surface of the diameter decreasing portion in the radial direction by the other one of the first member and the second member by moving the first member and the second member along the longitudinal direction of the elongated body by the rotation of the first member and the second member relative to each other.

20. The method according to claim 17, further comprising:
rotating the first member relative to the second member in a second direction, the second direction being a direction opposite to the first direction;
releasing the elongated body from being held by the diameter decreasing portion of the torque device; and
removing the elongated body from the torque device.
